(19) United States
(12) Patent Application Publication
(10) Pub. No.: US 2006/0058798 A1
(43) Pub. Date: Mar. 16, 2006

(54) Bone distractor with ratchet mechanism

(76) Inventors: Shawn D. Roman, Orange Park, FL (US); Ryan N. Luby, Jacksonville, FL (US); Saddy R. Garcia, St. Augustine, FL (US); Steven V. Palmer, Jacksonville, FL (US); Adam Hausman, Jacksonville, FL (US)

Correspondence Address: HARNESS, DICKEY & PIERCE, P.L.C., P.O. BOX 828, BLOOMFIELD HILLS, MI, 48303, US

(21) Appl. No.: 11/207,063

(22) Filed: Aug. 18, 2005

Related U.S. Application Data
(60) Provisional application No. 60/604,167, filed on Aug. 24, 2004.

(51) Int. Cl. A61B 17/80

(52) U.S. Cl. 606/71

(57) ABSTRACT

A bone distractor for distracting bone on opposite sides of an osteotomy of the bone include a first affixation member for affixation to the bone on one side of the osteotomy and a second affixation member for affixation to the bone on another side of the osteotomy. A drive screw has a rotatable member engaging the first and second affixation members for distracting the first and second affixation members relative to each other in response to rotation of the rotatable member. A ratchet mechanism selectively limits rotation of the drive screw in one direction.

### FIELD

The present teachings relate generally to an apparatus for correction of bone abnormalities, and more particularly to an apparatus for affixation to bones to effect distraction of the bones relative to a fracture or osteotomy.

### BACKGROUND

Various developmental disorders may result in bone abnormalities in which certain bones fail to grow in proper proportion to other bones, or in which certain bones fuse prematurely, causing malformation. Such abnormalities may be corrected by separating the bones through osteotomy or fracture, and then slowly distracting the bones relative to each other, thereby inducing bone growth at the separation line. As the bones are distracted, the newly formed bone at the growth surfaces adjacent the separation fills in the increasing gap between the bones.

An apparatus for effecting distraction may involve two affixation members, in which each affixation member is temporarily affixed to the bone on opposite sides of the osteotomy, such as by bone screws, and a drive screw that threadedly engages at least one of the affixation members. By rotating the drive screw incrementally and periodically, the two affixation members are driven apart, and hence the bones on either side of the osteotomy are distracted relative to the location of the osteotomy. Rotation of the drive screw in the wrong direction, however, may cause damage to the distractor, such as before affixation, or prevent distraction, such as in later stages of a procedure. This latter situation may occur when a patient manipulates the distractor improperly or micromovements and force from surrounding soft tissue force the two affixation members together along the drive screw.

A distractor including a mechanism to selectively restrict rotation of the drive screw in the wrong direction may limit damage to the distractor and promote efficacy of distraction.

### SUMMARY

A bone distractor is provided for distracting bone on opposite sides of an osteotomy of the bone. The bone distractor includes a first affixation member for affixation to the bone on one side of the osteotomy, and a second affixation member for affixation to the bone on another side of the osteotomy. A drive screw has a rotatable member that engages the first and second affixation members for distracting the first and second affixation members relative to each other in response to rotation of the rotatable member. A ratchet interacts with the rotatable member to allow rotation in one direction and selectively prevent rotation in another direction.

Further areas of applicability will become apparent from the description provided hereinafter. It should be understood that the description and examples are intended for purposes of illustration and are not intended to limit the scope of the teachings.

### BRIEF DESCRIPTION OF THE DRAWINGS

The present teachings will become more fully understood from the detailed description and the accompanying drawings, wherein:

FIG. 1 is a perspective view of a bone distractor according to the teachings;

FIG. 2 is an exploded view of the bone distractor of FIG. 1;

FIG. 3 is a sectional view of the bone distractor of FIG. 1 along line 3-3 with a release pin removed;

FIG. 4 is a sectional view of the bone distractor of FIG. 3 with a release pin engaged;

FIG. 5 is a perspective view of a bone distractor according to the teachings;

FIG. 6 is an exploded view of the bone distractor of FIG. 4;

FIG. 7 is a sectional view of the bone distractor of FIG. 4 along line 7-7 with a release pin removed;

FIG. 8 is a sectional view of the bone distractor of FIG. 7 with a release pin engaged;

FIG. 9 is a perspective view of a bone distractor according to the teachings;

FIG. 10 is an exploded view of the bone distractor of FIG. 9;

FIG. 11 is a sectional view of the bone distractor of FIG. 9 along line 11-11 with a release pin removed;

FIG. 12 is a sectional view of the bone distractor of FIG. 11 with a release pin engaged;

FIG. 13 is a perspective view of a bone distractor according to the teachings;

FIG. 14 is an exploded view of the bone distractor of FIG. 13;

FIG. 15 is a sectional view of the bone distractor of FIG. 13 along line 15-15 with a release pin removed;

FIG. 16 is a sectional view of the bone distractor of FIG. 15 with a release pin engaged;

FIG. 17 is a perspective view of a bone distractor according to the teachings;

FIG. 18 is an exploded view of the bone distractor of FIG. 17;

FIG. 19 is a sectional view of the bone distractor of FIG. 17 along line 19-19 in a lock position;

FIG. 20 is a sectional view of the bone distractor of FIG. 19 in a ratchet position;

FIG. 21 is a sectional view of the bone distractor of FIG. 19 in an open position;

FIG. 22 is a perspective view of a bone distractor according to the teachings;

FIG. 23 is an exploded view of the bone distractor of FIG. 22;

FIG. 24 is a sectional view of the bone distractor of FIG. 22 along line 24-24 with a release pin removed;

FIG. 25 is a sectional view of the bone distractor of FIG. 24 with a release pin engaged;

FIG. 26a is a perspective view of a flex tube according to the present teachings;

FIG. 26b is a detail view of FIG. 26a;

FIG. 26c is a sectional view of the bone distractor of FIG. 26A along line 26c-26c;

FIG. 27 is a sectional view of the bone distractor of FIG. 22 along line 27-27;

FIG. 28 is a perspective view of a bone distractor according to the teachings;

FIG. 29 is a sectional view of the bone distractor of FIG. 28 along line 29-29; and

FIG. 30 is a sectional view of the bone distractor of FIG. 29 shown with a drive screw rotating.

### DESCRIPTION

The following description is merely exemplary in nature and is in no way intended to limit the teachings, its application, or uses.

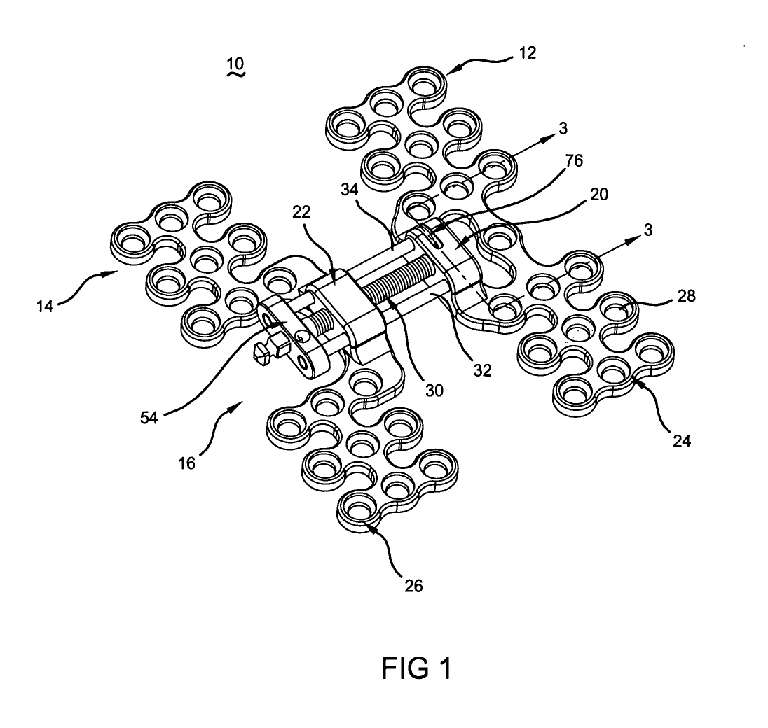

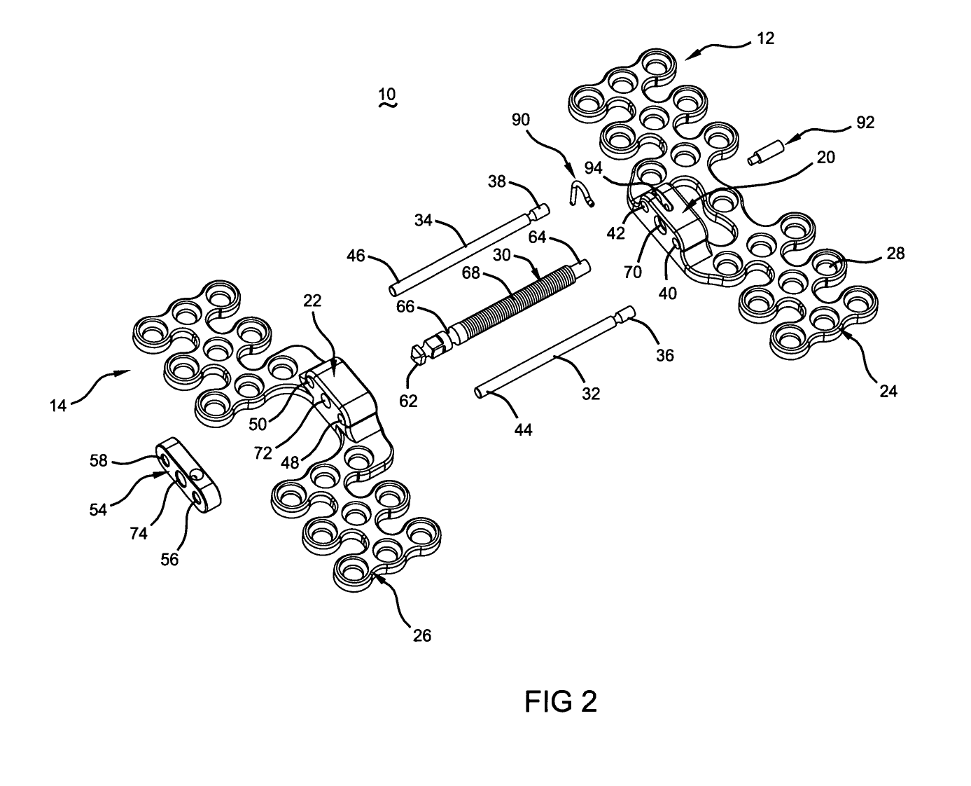

As illustrated in FIGS. 1-4, a bone distractor 10 may include a first affixation member 12, a second affixation member 14, and an adjustment assembly 16. The first affixation member 12 and the second affixation member 14 are moveable relative one another through the adjustment assembly 16.

The first affixation member 12 and the second affixation member 14 may include a central bridge portion 20, 22, respectively, and wing elements 24, 26, respectively, which may extend transversely from the respective central bridge portion 20, 22. Wing elements 24, 26 may be generally planar and may include multiple screw holes 28 operable to receive bone screws to secure wing elements 24, 26 to bone, wherein wing elements 24, 26 may lie flat adjacent to the applied bone surface.

The adjustment assembly 16 may include a drive screw 30, a pair of rods 32, 34, a ratchet mechanism 76 and a block 54. The drive screw 30 and rods 32, 34 may be disposed adjacent and parallel to one another. One end of rods 32, 34 may be secured within corresponding holes in bridge portion 20 of the first affixation member 12 and an opposite end may be slidably received through corresponding holes through bridge portion 22 of the second affixation member 14 and secured within corresponding holes in the block 54. More specifically, first ends 36, 38 of rods 32, 34, respectively, may be received by holes 40, 42 in bridge portion 20 of first affixation member 12. Second ends 44, 46 of rods 32, 34, respectively, may be received through corresponding holes 48, 50 in bridge portion 22 of second affixation member 14 and terminate in holes 56, 58 of the block 54.

The drive screw 30 may include a drive head 62 at one end and a tip 64 at an opposite end. Between the drive head 62 and the tip 64 is a non-threaded portion 66 and a threaded portion 68. The drive head 62 may be driven by a tool. The tip 64 may be received within an aperture 70 in the bridge portion 20. The threaded portion 68 may be received through a threaded aperture 72 in the bridge portion 22. The non-threaded portion 66 may be received through an aperture 74 in the block 54.

The tip 64 may be rotably mounted in aperture 70 of block 20 of first affixation member 12 and may be selectively restricted from rotating in one direction by the ratchet mechanism 76. The tip 64 may include a groove 80 formed longitudinally in an outer surface. The groove 80 may include a first wall 82 and a second wall 84. The first wall 82 may be shorter than the second wall 84.

The ratchet mechanism 76 may be disposed within the bridge portion 20 of first affixation member 12 and may include a spring 90 and a release pin 92. The spring 90 may be disposed in a channel 94 formed in bridge portion 20, abut end 36 of rod 32 in aperture 40, and may generally extend between aperture 40 and aperture 70. An anchor end 96 of the spring 90 may be press fit in an aperture formed in bridge portion 20 and the free end 98 of the spring 90 may selectively extend into groove 80 of tip 64.

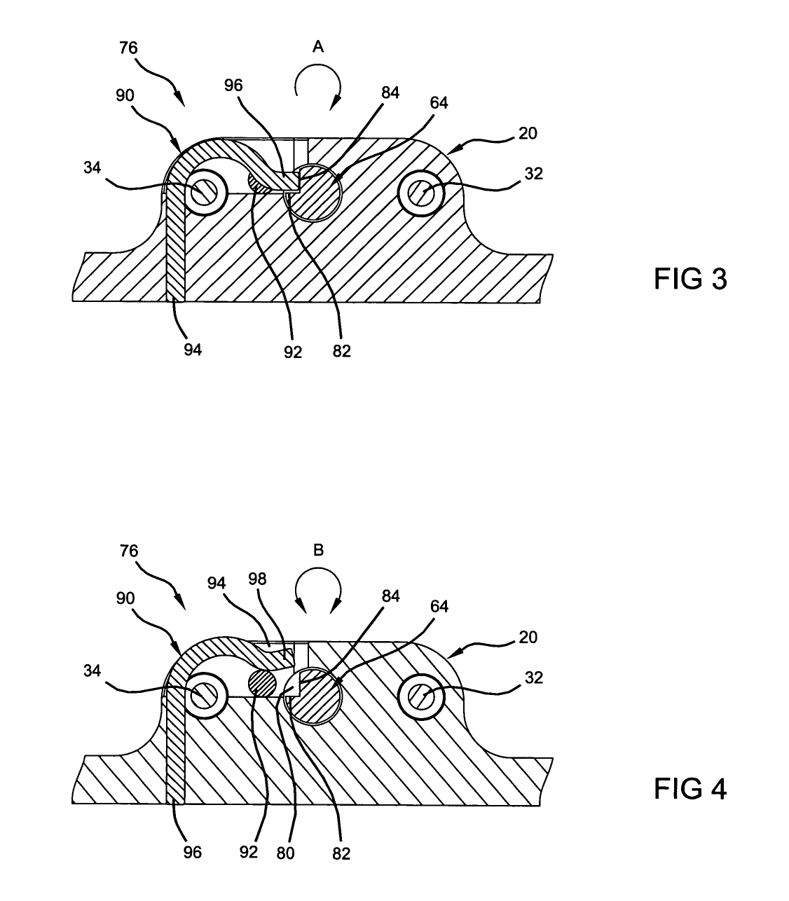

When drive screw 30 is rotated in a first direction illustrated by Arrow A in FIG. 3, the free end 96 of the spring 90 may ride along first wall 82 of the groove 80, thereby deflecting the free end 96 of the spring 90 and permitting rotation of the drive screw 30. When rotation in an opposite direction is attempted, the free end 96 of the spring 90 may engage the lateral wall 84 of the groove 80, thereby preventing rotation. Thus, rotation in the direction opposite Arrow A is not permitted due to blocking engagement of second wall 84 and free end 96 of spring 90. Insertion of the release pin 92 forces the free end 96 of the spring 90 out of engagement with the groove 80 of tip 64, thereby allowing rotation in either direction, as illustrated by Arrow B in FIG. 4.

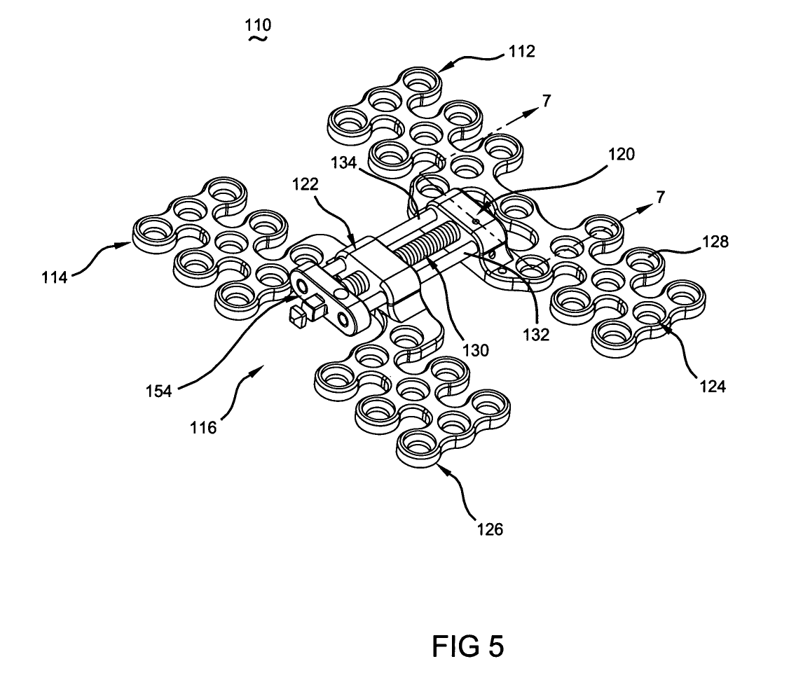

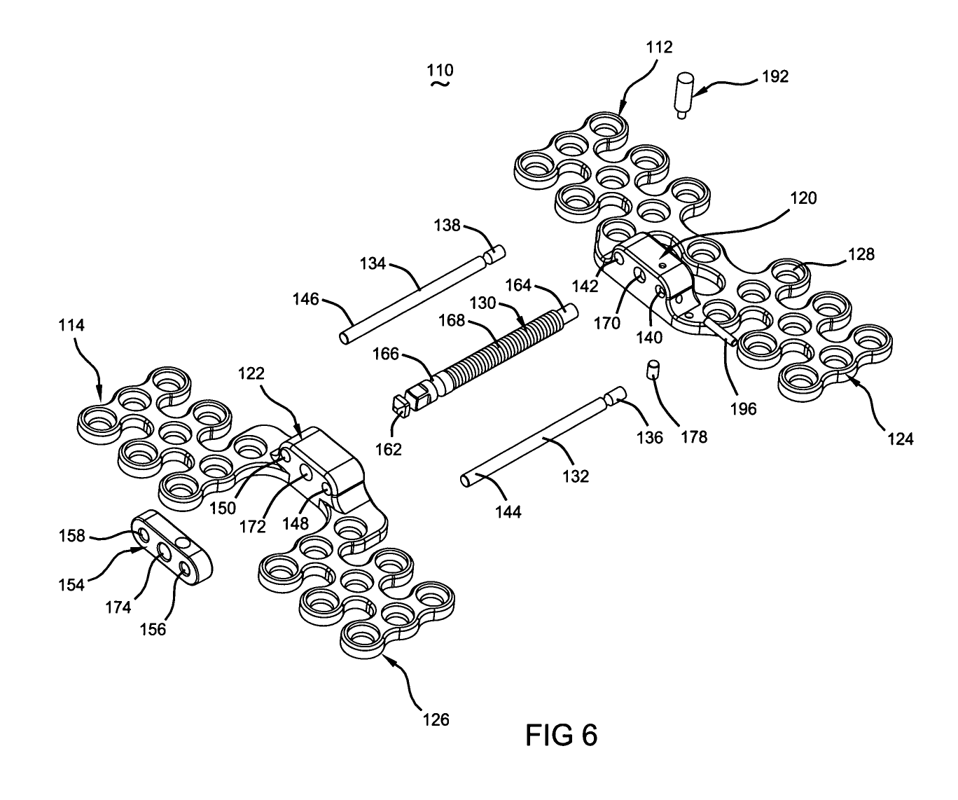

As illustrated in FIGS. 5-8, a bone distractor 110 may include a first affixation member 112, a second affixation member 114, and an adjustment assembly 116. The first affixation member 112 and the second affixation member 114 are moveable relative one another through the adjustment assembly 116.

The first affixation member 112 and the second affixation member 114 may include a central bridge portion 120, 122, respectively, and wing elements 124, 126, respectively, which may extend transversely from the respective central bridge portion 120, 122. Wing elements 124, 126 may be generally planar and may include multiple screw holes 128 operable to receive bone screws to secure wing elements 124, 126 to bone, wherein wing elements 124, 126 may lie flat adjacent to the applied bone surface.

The adjustment assembly 116 may include a drive screw 130, a pair of rods 132, 134, a ratchet mechanism 176 and a block 154. The drive screw 130 and rods 132, 134 may be disposed adjacent and parallel to one another. One end of rods 132, 134 may be secured within corresponding holes in bridge portion 120 of the first affixation member 112 and an opposite end may be slidably received through corresponding holes through bridge portion 122 of the second affixation member 114 and secured within corresponding holes in the block 154. More specifically, first ends 136, 138 of rods 132, 134, respectively, may be received by holes 140, 142 in bridge portion 120 of first affixation member 112. Second ends 144, 146 of rods 132, 134, respectively, may be received through corresponding holes 148, 150 in bridge portion 122 of second affixation member 114 and terminate in holes 156,158 of the block 154.

The drive screw 130 may include a drive head 162 at one end and a tip 164 at an opposite end. Between the drive head 162 and the tip 164 is a non-threaded portion 166 and a threaded portion 168. The drive head 162 may be driven by a tool. The tip 164 may be received within an aperture 170 in the bridge portion 120. The threaded portion 168 may be received through a threaded aperture 172 in the bridge portion 122. The non-threaded portion 166 may be received through an aperture 174 in the block 154.

The tip 164 may be rotably mounted in aperture 170 of bridge portion 120 of first affixation member 112 and may be selectively restricted from rotating in one direction by the ratchet mechanism 176. The tip 164 may include a groove 180 formed longitudinally in an outer surface. The groove 180 may include a first wall 182 and a second wall 184. The first wall 182 may be shorter than the second wall 184.

The ratchet mechanism 176 may be disposed within the bridge portion 120 of first affixation member 112 and may include a spring 190 and a release pin 192. The spring 190 may be disposed in a channel 194 formed in bridge portion 120, abut end 136 of rod 132 in aperture 140, and may generally extend between aperture 140 and aperture 170. An anchor end 196 of the spring 190 may be press fit in an aperture formed in bridge portion 122 and the free end 198 of the spring 190 may selectively extend into groove 180 of tip 164.

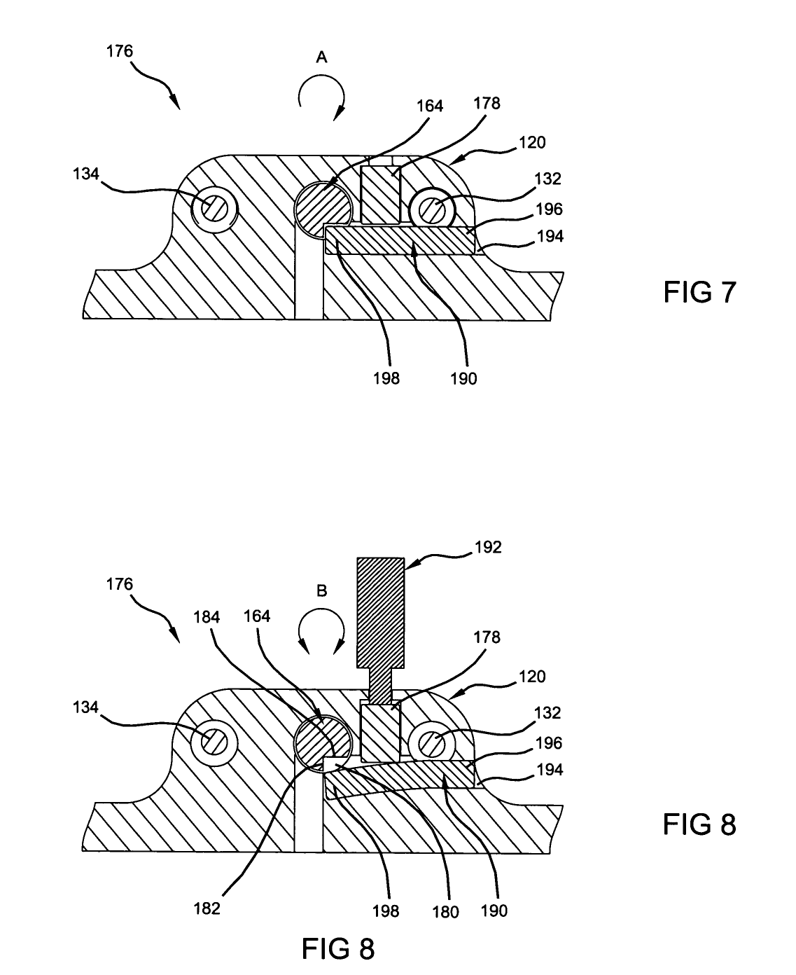

When drive screw 130 is rotated in a first direction illustrated by Arrow A in FIG. 7, the free end 198 of the spring 190 is contacted by lateral wall 184 of the groove 180, thereby deflecting the free end 198 of the spring 190 and permitting rotation of the drive screw 130. When rotation in an opposite direction is attempted, the free end of the spring engages the first wall 182, thereby preventing rotation. Thus, rotation in the direction opposite Arrow A is not permitted due to blocking engagement of first wall 182 and free end 198 of spring 190. Insertion of the release pin 192 impacts block 178, which moves into contact with the free end 198 of the spring 190, thereby forcing the spring 190 out of engagement with the groove 180 of tip 164. In this position, the drive screw 130 may rotate in either direction, as illustrated by Arrow B in FIG. 8.

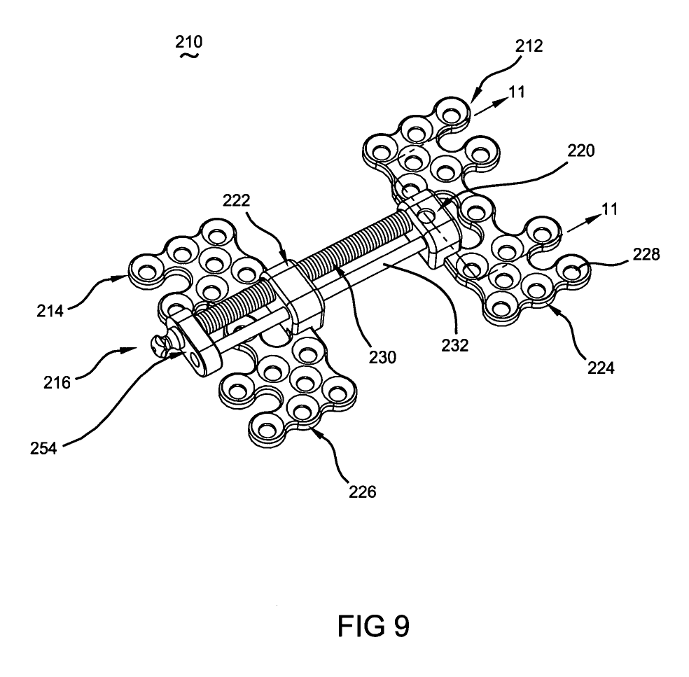

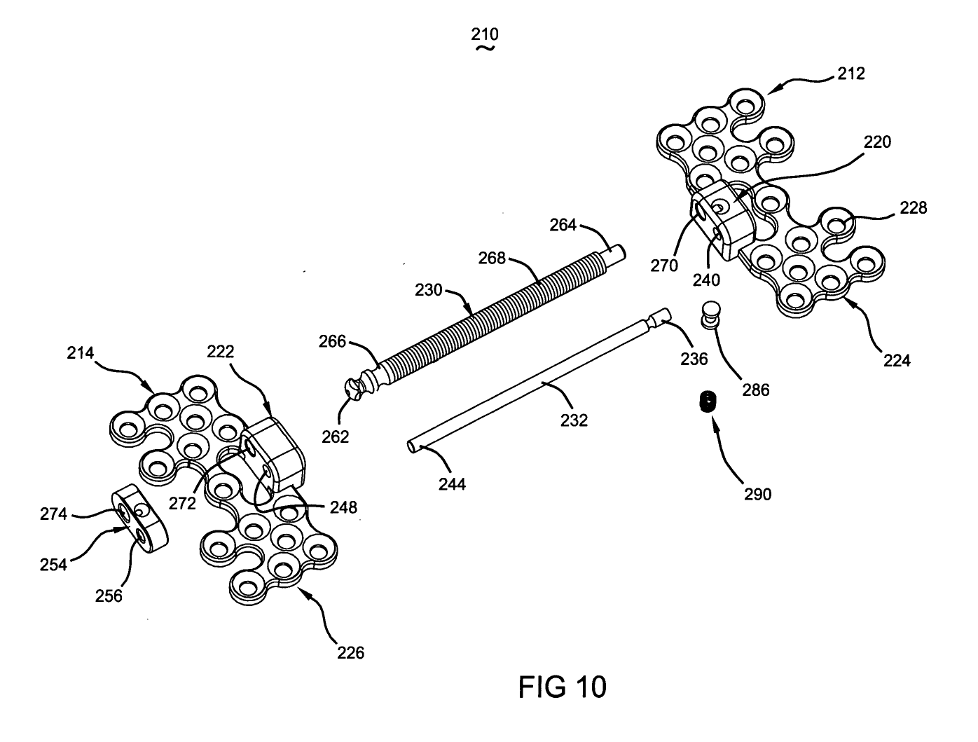

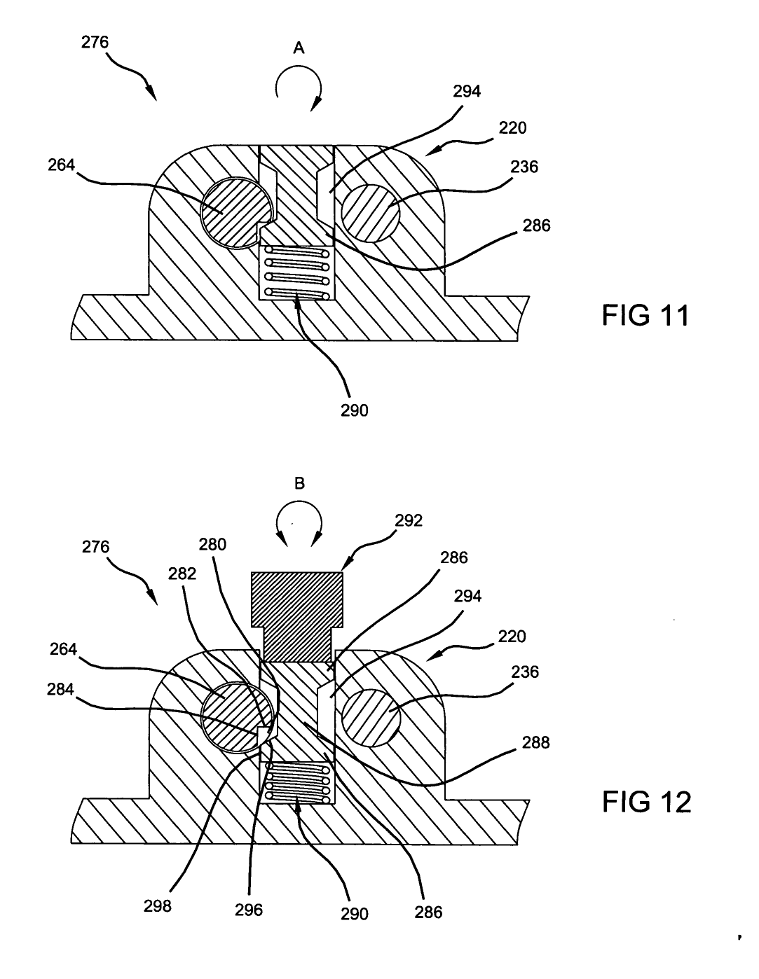

As illustrated in FIGS. 9-12, a bone distractor 210 may include a first affixation member 212, a second affixation member 214, and an adjustment assembly 216. The first affixation member 212 and the second affixation member 214 are moveable relative one another through the adjustment assembly 216.

The first affixation member 212 and the second affixation member 214 may each include a central bridge portion 220, 222, respectively, and wing elements 224, 226, respectively, which may extend transversely from the respective central bridge portion 220, 222. Wing elements 224, 226 may be generally planar and may include multiple screw holes 228 operable to receive bone screws to secure wing elements 224, 226 to bone, wherein wing elements 224, 226 may lie flat adjacent to the applied bone surface.

The adjustment assembly 216 may include a drive screw 230, a rod 232, a ratchet mechanism 276 and a block 254. The drive screw 230 and rod 232 may be disposed adjacent and parallel to one another. One end of rod 232 may be secured within a corresponding hole in bridge portion 220 of the first affixation member 212 and an opposite end may be slidably received through a corresponding hole through bridge portion 222 of the second affixation member 214 and secured within a corresponding hole in block 254. More specifically, first end 236 of rod 232 may be received by hole 240 in bridge portion 222 of first affixation member 212. Second end 244 of rod 232 may be received through corresponding hole 248 in bridge portion 222 of second affixation member 214 and terminate in hole 256 of the block 254.

The drive screw 230 may include a drive head 262 at one end and a tip 264 at an opposite end. Between the drive head 262 and the tip 264 is a non-threaded portion 266 and a threaded portion 268. The drive head 262 may be driven by a tool. The tip 264 may be received within an aperture 270 in the bridge portion 220. The threaded portion 268 may be received through a threaded aperture 272 in the bridge portion 222. The non-threaded portion 266 may be received through an aperture 274 in the block 254.

The tip 264 may be rotably mounted in aperture 270 of block 220 of first affixation member 212 and may be selectively restricted from rotating in one direction by the ratchet mechanism 276. The tip 264 may include a groove 280 formed longitudinally in an outer surface. The groove 280 may include a first wall 282 and a second wall 284. The first wall 282 may be shorter than the second wall 284.

The ratchet mechanism 276 is disposed within the bridge portion 220 of first affixation member 212 and may include a spring 290, a release pin 292, and a ratchet pin 288. The ratchet pin 288 includes a body connecting bulbous ends 286, which include a cam surface 296 and a blocking surface 298. The spring 290 and ratchet pin 288 may be disposed in a channel 294 formed in bridge portion 220, and may be generally located between aperture 240 and aperture 270. The spring 290 biases the ratchet pin 288 into a ratchet position wherein one end 286 engages tip 264.

When drive screw 230 is rotated in a first direction illustrated by Arrow A in FIG. 3, the end 286 of the ratchet pin 288 engages first wall 282 of the groove 280. The cam surface 296 of ratchet pin 288 forces the ratchet pin 288 against the bias of spring 290, thereby permitting rotation of the drive screw 230. When rotation in an opposite direction is attempted, the second wall 284 engages the blocking surface 298, thereby preventing rotation. Thus, rotation in the direction opposite Arrow A is not permitted due to blocking engagement of second wall 284 and blocking surface 298. Insertion of the release pin 292 forces the ratchet pin 288 against the bias of spring 290 and out of engagement with the groove 280 of tip 264, thereby allowing rotation in either direction, as illustrated by Arrow B in FIG. 4.

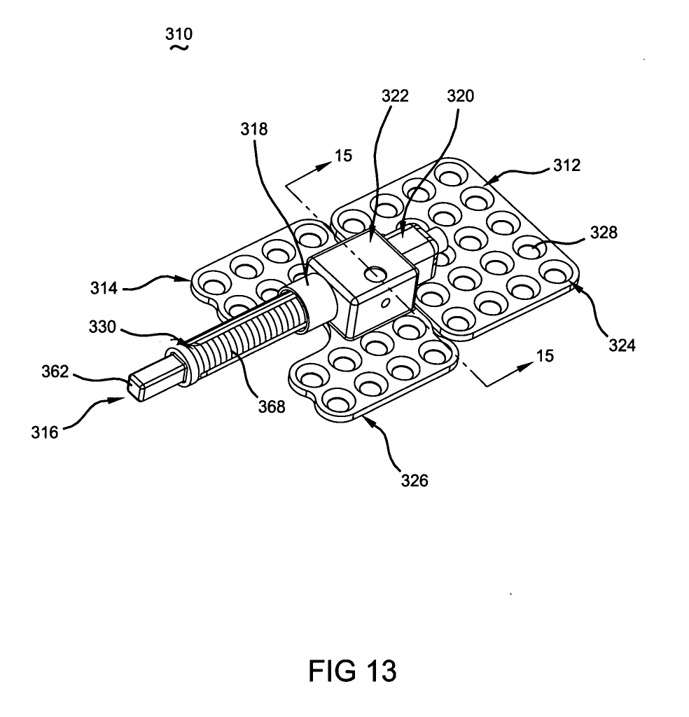

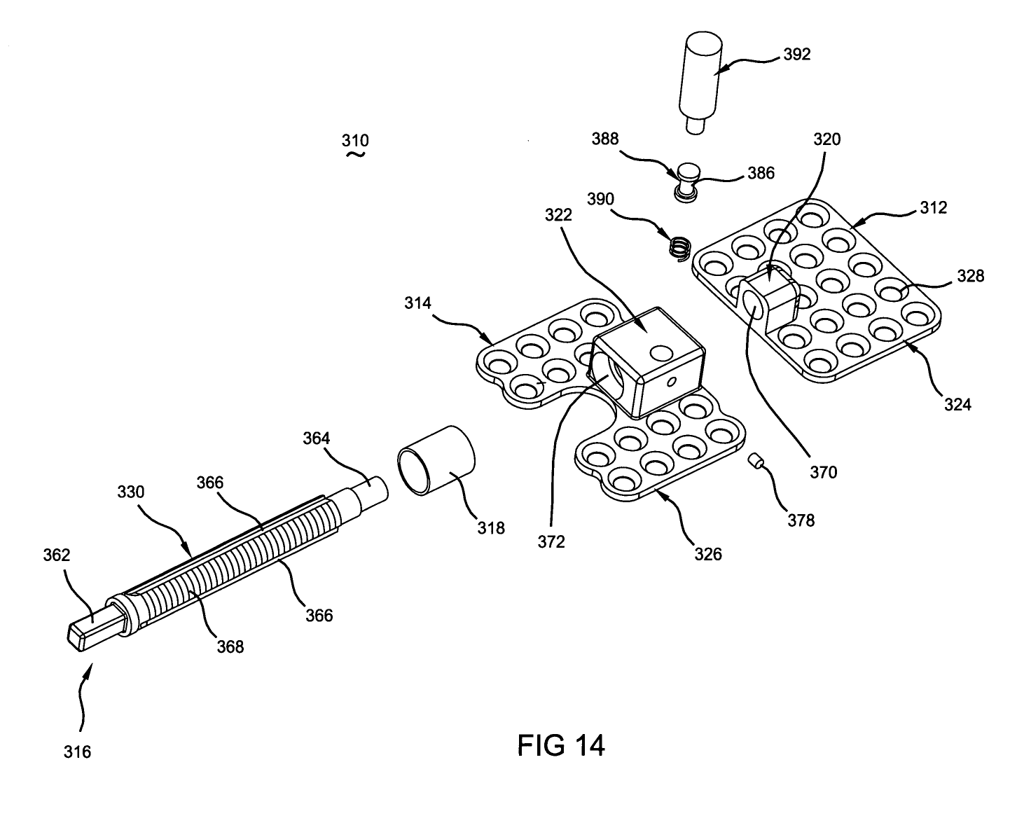

As illustrated in FIGS. 13-16, a bone distractor 310 may include a first affixation member 312, a second affixation member 314, and an adjustment assembly 316. The first affixation member 312 and the second affixation member 314 are moveable relative one another through the adjustment assembly 316.

The first affixation member 312 and the second affixation member 314 may include a central bridge portion 320, 322, respectively, and wing elements 324, 326, respectively, which may extend transversely from the respective central bridge portion 320, 322. Wing elements 324, 326 may be generally planar and may include multiple screw holes 328 operable to receive bone screws to secure wing elements 324, 326 to bone, wherein wing elements 324, 326 may lie flat adjacent to the applied bone surface. Central bridge portion 322 includes a sleeve 318.

The adjustment assembly 316 may include a drive screw 330 and a ratchet mechanism 376. The drive screw 330 may include a drive head 362 at one end and a tip 364 at an opposite end. Between the drive head 362 and the tip 364 is a threaded portion 368 and equally spaced apart longitudinal grooves 366. The drive head 362 may be driven by a tool. The tip 364 may be received within an aperture 370 in the bridge portion 320. The threaded portion 368 and grooves 366 may be received through a threaded aperture 372 in the bridge portion 322. The grooves 366 engage the ratchet mechanism 376.

The tip 364 may be rotably mounted in aperture 370 of block 320 of first affixation member 312. The threaded portion 368 may be threadably mounted in aperture 372 of block 322 of second affixation member 314 and may be selectively restricted from rotating in one direction by the ratchet mechanism 376. The grooves 366 may include a first wall 382 and a second wall 384. The first wall 382 may be shorter than the second wall 384.

The ratchet mechanism 376 is disposed within bridge portion 322 of first affixation member 312 and may include a spring 390, a release pin 392, and a ratchet pin 388. The ratchet pin 388 includes a body connecting bulbous ends 386, which include a cam surface 396 and a blocking surface 398. The spring 390 and ratchet pin 388 may be disposed in a channel 394 formed in bridge portion 322. The spring 390 biases the ratchet pin 388 into a ratchet position wherein one end 386 engages a groove 366. A stop 378 may extend into the channel 394 between ends 386 of ratchet pin 388 to limit travel of the ratchet pin 388 within the channel 394.

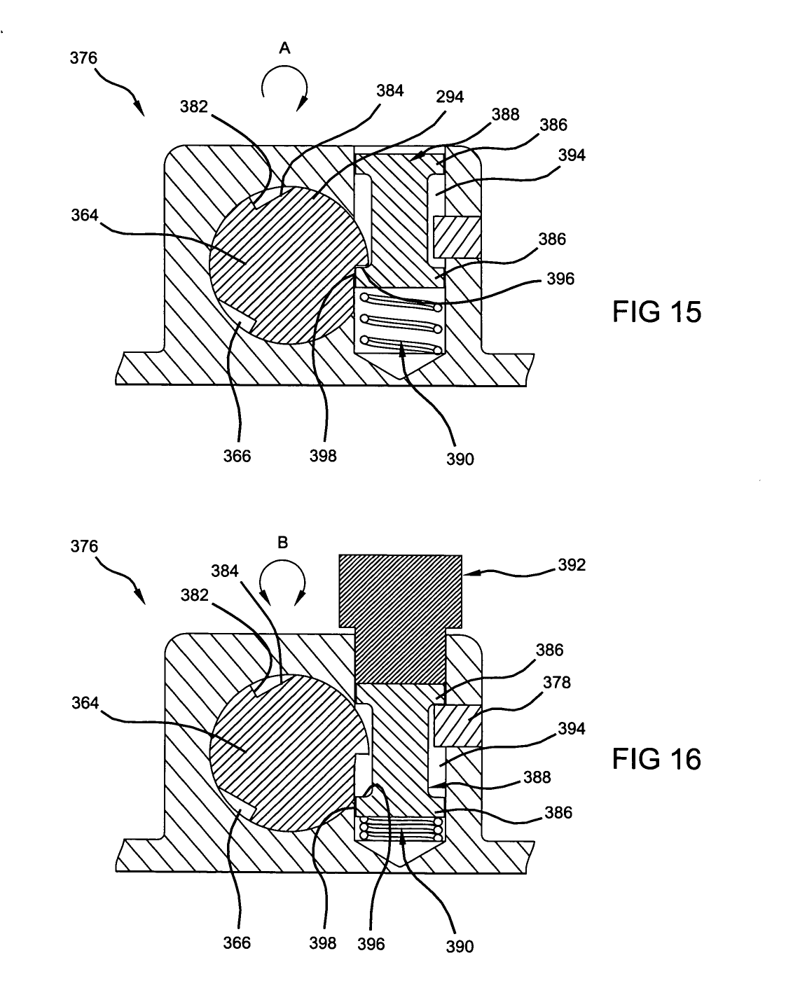

When drive screw 330 is rotated in a first direction illustrated by Arrow A in FIG. 15, the end 386 of the ratchet pin 388 engages first wall 382 of groove 366. The cam surface 396 of ratchet pin 388 forces the ratchet pin 388 against the bias of spring 390, thereby permitting rotation of the drive screw 330. When rotation in an opposite direction is attempted, the second wall 384 engages the blocking surface 398, thereby preventing rotation. Thus, rotation in the direction opposite Arrow A is not permitted due to blocking engagement of second wall 384 and blocking surface 398. Insertion of the release pin 392 forces the ratchet pin 388 against the bias of spring 390 and out of engagement with the groove 380 of tip 364, thereby allowing rotation in either direction, as illustrated by Arrow B in FIG. 4.

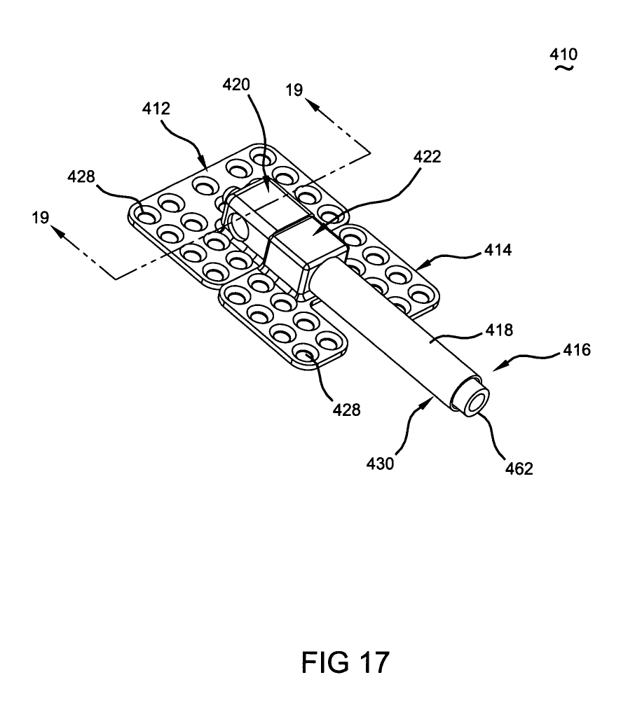

As illustrated in FIGS. 17-21, a bone distractor 410 may include a first affixation member 412, a second affixation member 414, and an adjustment assembly 416. The first affixation member 412 and the second affixation member 414 are moveable relative one another through the adjustment assembly 416.

The first affixation member 412 and the second affixation member 414 may include a central bridge portion 420, 422, respectively, and wing elements 424, 426, respectively, which may extend transversely from the respective central bridge portion 420, 422. Wing elements 424, 426 may be generally planar and include multiple screw holes 428 operable to receive bone screws to secure wing elements 424, 426 to bone, wherein wing elements 424, 426 may lie flat adjacent to the applied bone surface. Bridge portion 422 includes a sleeve 418.

The adjustment assembly 416 may include a drive screw 430 and a ratchet mechanism 476. The drive screw 430 may include a drive head 462 at one end and a tip 464 at an opposite end. Between the drive head 462 and the tip 464 is a non-threaded portion 466 and a threaded portion 468. The drive head 462 may be driven by a tool. The tip 464 and non-threaded portion 466 may be received within an aperture 470 in the bridge portion 420. The threaded portion 468 may be received through a threaded aperture 472 in the bridge portion 422.

The tip 464 and non-threaded portion 466 may be rotably mounted in aperture 470 of block 420 of first affixation member 412 and may be selectively restricted from rotating in one direction by the ratchet mechanism 476. The non-threaded portion may include a pair of equally spaced-apart grooves 480 formed longitudinally in an outer surface 474. The grooves 480 may include a first wall 482 and a second wall 484 meeting at a corner 478. The first wall 482 may be shorter than the second wall 484.

The ratchet mechanism 476 may be disposed within the bridge portion 420 of first affixation member 412 and may include a spring 490, a release pin 492, and a ratchet pin 488. The ratchet pin 488 includes a body having a keyed aperture 486 therethrough and a channel 436 formed on an outer surface. Within the aperture 486, the ratchet pin 488 includes a cam surface 496 and a blocking surface 498. The spring 490 and ratchet pin 488 may be disposed in a channel 494 formed in bridge portion 420. The spring 490 biases the ratchet pin 488 into a ratchet position wherein one end 486 engages top 464. A stop pin 434 may be positioned in bridge portion 420 through channel 494 therethrough and channel 436 of ratchet pin 488 to limit travel of ratchet pin 488 in channel 494.

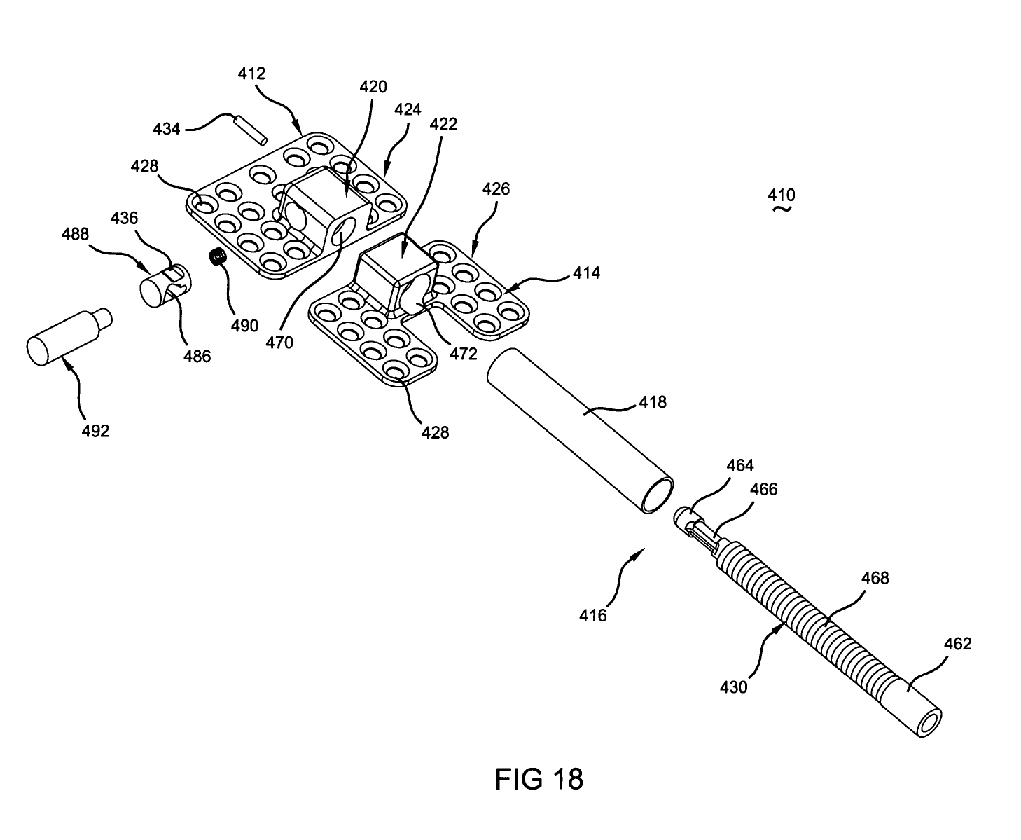

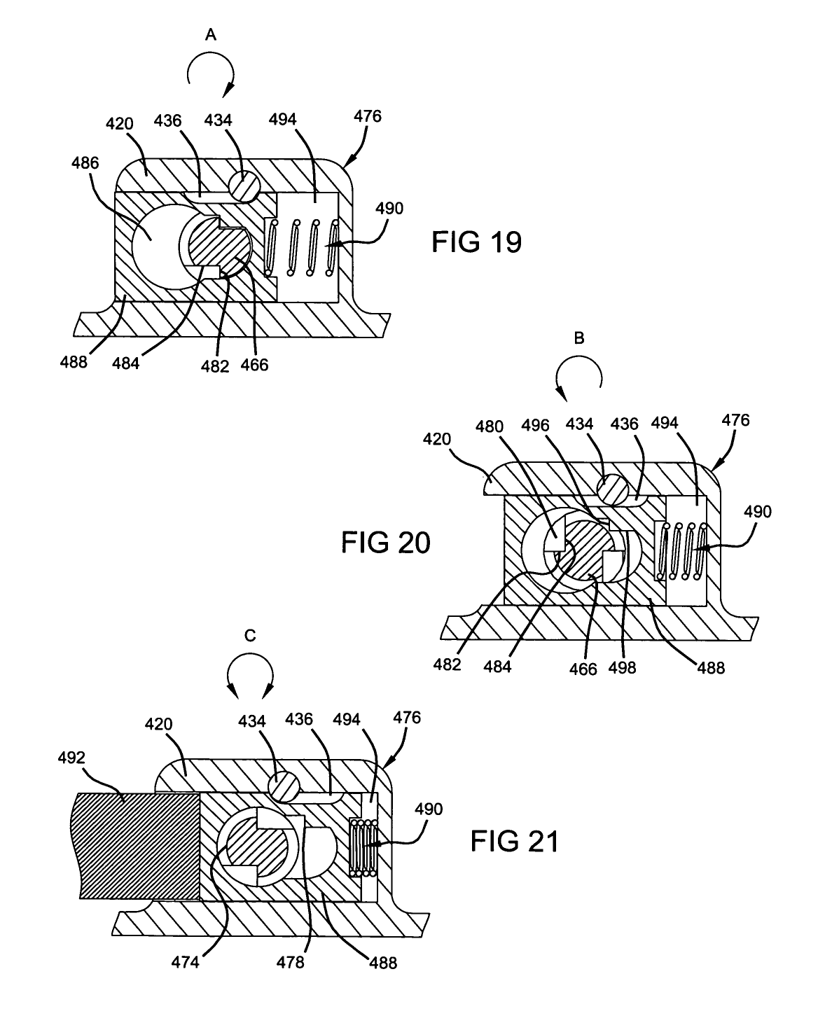

When drive screw 430 is rotated in a first direction illustrated by Arrow A in FIG. 19, the ratchet pin 488 engages first wall 482 of the groove 480. The cam surface 496 of ratchet pin 488 forces the ratchet pin 488 against the bias of spring 490, thereby permitting rotation of the drive screw 430, as illustrated between FIGS. 19 and 20. Once rotated sufficiently to slide cam surface 496 out of groove 480, the spring 490 forces corner 478 to ride along surface 474 of non-threaded portion 466 until engaging another groove 480, wherein cam surface 496 again forces ratchet pin 488 against the bias of spring 490. When rotation in an opposite direction is attempted, the second wall 484 engages the blocking surface 498, thereby preventing rotation. Thus, from the position illustrated in FIG. 19, rotation in the direction opposite Arrow A is not permitted due to blocking engagement of second wall 484 and blocking surface 498. From the position illustrated in FIG. 20, limited rotation in the direction of Arrow B would be possible until groove 80 seats corner 478. Insertion of the release pin 492, as shown in FIG. 21, forces the ratchet pin 488 against the bias of spring 490 and out of engagement with the groove 480 of tip 464, thereby allowing rotation in either direction, as illustrated by Arrow C.

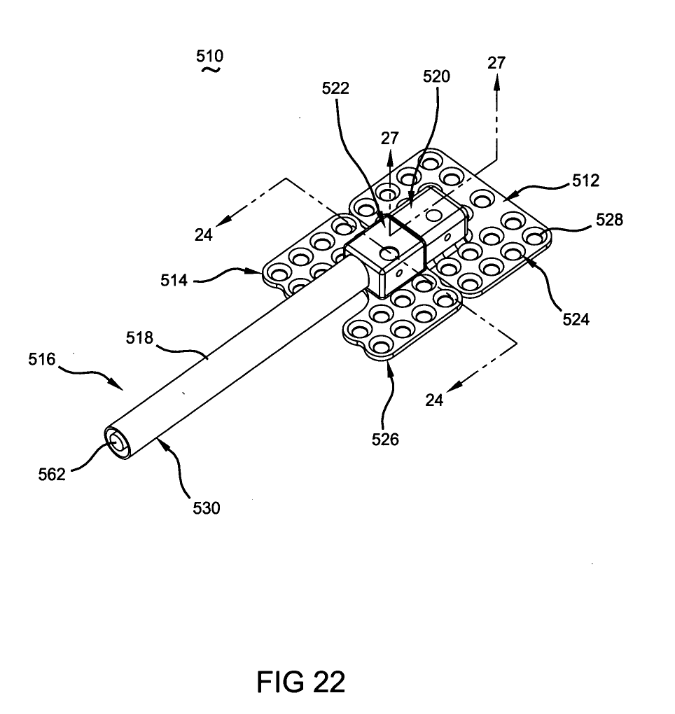

As illustrated in FIGS. 22-27, a bone distractor 510 may include a first affixation member 512, a second affixation member 514, and an adjustment assembly 516. The first affixation member 512 and the second affixation member 514 are moveable relative one another through the adjustment assembly 516.

The first affixation member 512 and the second affixation member 514 may include a central bridge portion 520, 522, respectively, and wing elements 524, 526, respectively, which may extend transversely from the respective central bridge portion 520, 522. Wing elements 524, 526 may be generally planar and include multiple screw holes 528 operable to receive bone screws to secure wing elements 524, 526 to bone, wherein wing elements 524, 526 may lie flat adjacent to the applied bone surface. Bridge portion 522 includes a first flexible sleeve 518.

The adjustment assembly 516 may include a drive screw 530 and a ratchet mechanism 576. The drive screw 530 may include a drive head 562 at one end and a tip 564 at an opposite end. Between the drive head 562 and the tip 564 is a non-threaded portion 566 and a threaded portion 568, and equally spaced apart longitudinal grooves 580. The drive head 562 may be driven by a tool or connected to a flex tube and drive end. The tip 564 and non-threaded portion 566 may be received within an aperture 570 in the bridge portion 520. The threaded portion 568 and grooves 580 may be received through a threaded aperture 572 in the bridge portion 522.

The tip 564 and non-threaded portion 566 may be rotatably mounted in aperture 570 of block 520 of first affixation member 512 and may be selectively restricted from rotating in one direction by the ratchet mechanism 576. The grooves 580 may include a first wall 582 and a second wall. The first wall 582 may be shorter than the second wall 584.

The ratchet mechanism 576 is disposed within bridge portion 522 of first affixation member 512 and may include a spring 590, a release pin 592, and a ratchet pin 588. The ratchet pin 588 includes a body connecting bulbous ends 586, which include a cam surface 596 and a blocking surface 598. The spring 590 and ratchet pin 588 may be disposed in a channel 594 formed in bridge portion 522. The spring 590 biases the ratchet pin 588 into a ratchet position wherein one end 586 engages a groove 580. A stop 578 may extend into the channel 594 between ends 586 of ratchet pin 588 to limit travel of the ratchet pin 588 within the channel 594.

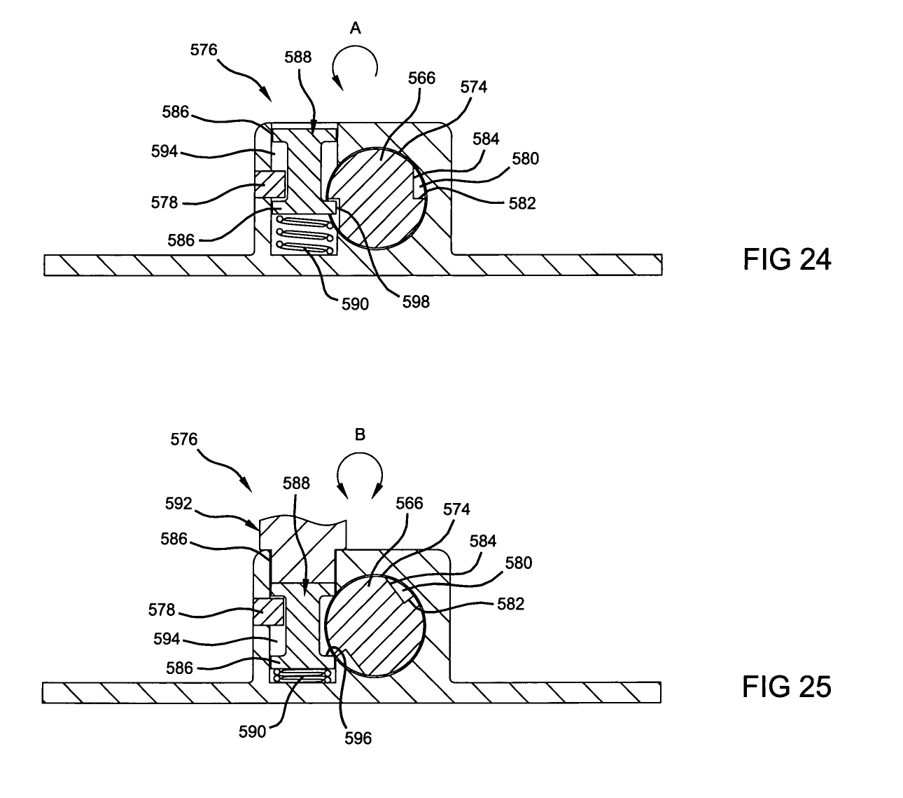

When drive screw 530 is rotated in a first direction illustrated by Arrow A in FIG. 24, the end 586 of the ratchet pin 588 engages first wall 582 of groove 580. The cam surface 596 of ratchet pin 588 forces the ratchet pin 588 against the bias of spring 590, thereby permitting rotation of the drive screw 530. When rotation in an opposite direction is attempted, the second wall 584 engages the blocking surface 598, thereby preventing rotation. Thus, rotation in the direction opposite Arrow A is not permitted due to blocking engagement of second wall 584 and blocking surface 598. Insertion of the release pin 592 forces the ratchet pin 588 against the bias of spring 590 and out of engagement with the groove 580, thereby allowing rotation in either direction, as illustrated by Arrow B in FIG. 25.

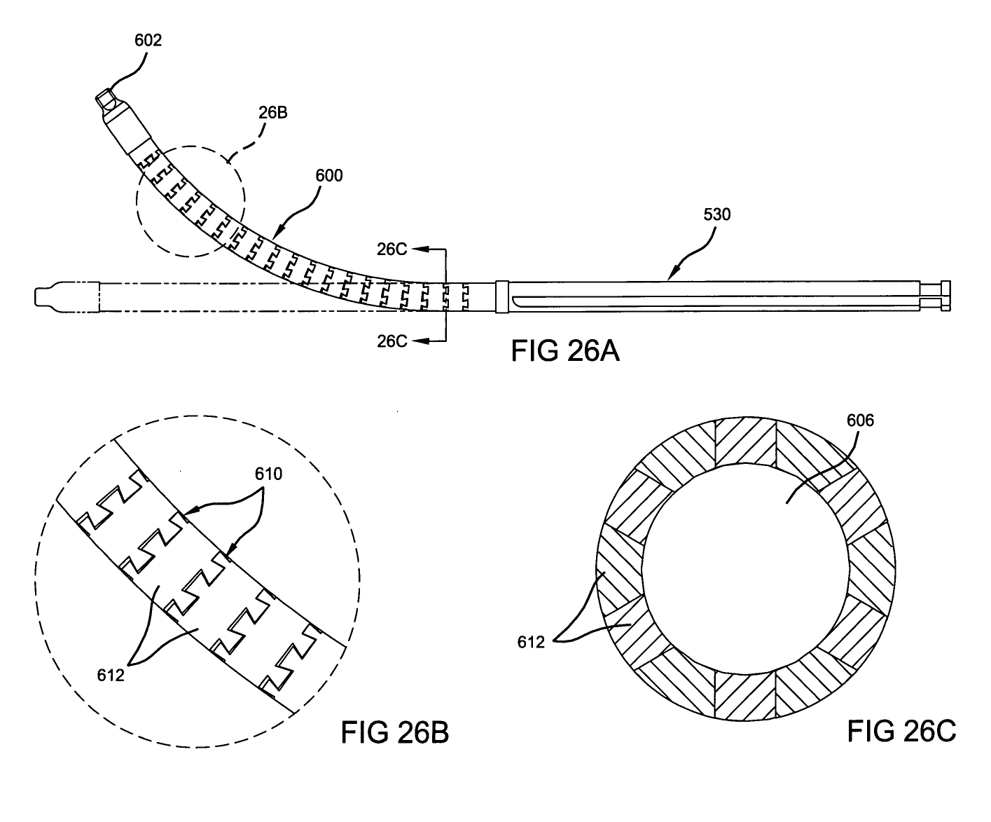

With reference to FIGS. 26a and 26b a longitudinal extension or flex tube 600 is shown attached to the drive screw 530. The flex tube 600 includes an interface head 602 for coupling with a driver (not shown). The flex tube 600 transmits torque from the driver to the drive screw 530. The flex tube 600 defines a cannulation 606 and may be formed of biocompatible metal such as titanium, stainless steel, cobalt chrome alloy or other materials. Alternatively, the flex tube may be formed of biocompatible plastic. In one example the biocompatible plastic may be resorbable such as Lactosorb offered by Biomet, Inc. of Warsaw, Ind.

The flex tube 600 defines a series of overlapping discontinuities 610 that allow the flex tube 600 to flex. More specifically, a series of interlocking links 612 are defined between adjacent discontinuities 610. Each interlocking link 612 is connected to an adjacent link 612 and allowed to provide an amount of lateral separation at the discontinuities 610 when the flex tube 600 is manipulated as illustrated in FIGS. 26a and 26b. The interlocking links 612 define dovetail sections at the discontinuities 610. Other patterns may similarly be employed. In one method of manufacturing the flex tube 600, the discontinuities 610 are cut in an interlocking pattern such as by a laser, thereby forming the interlocking links 612.

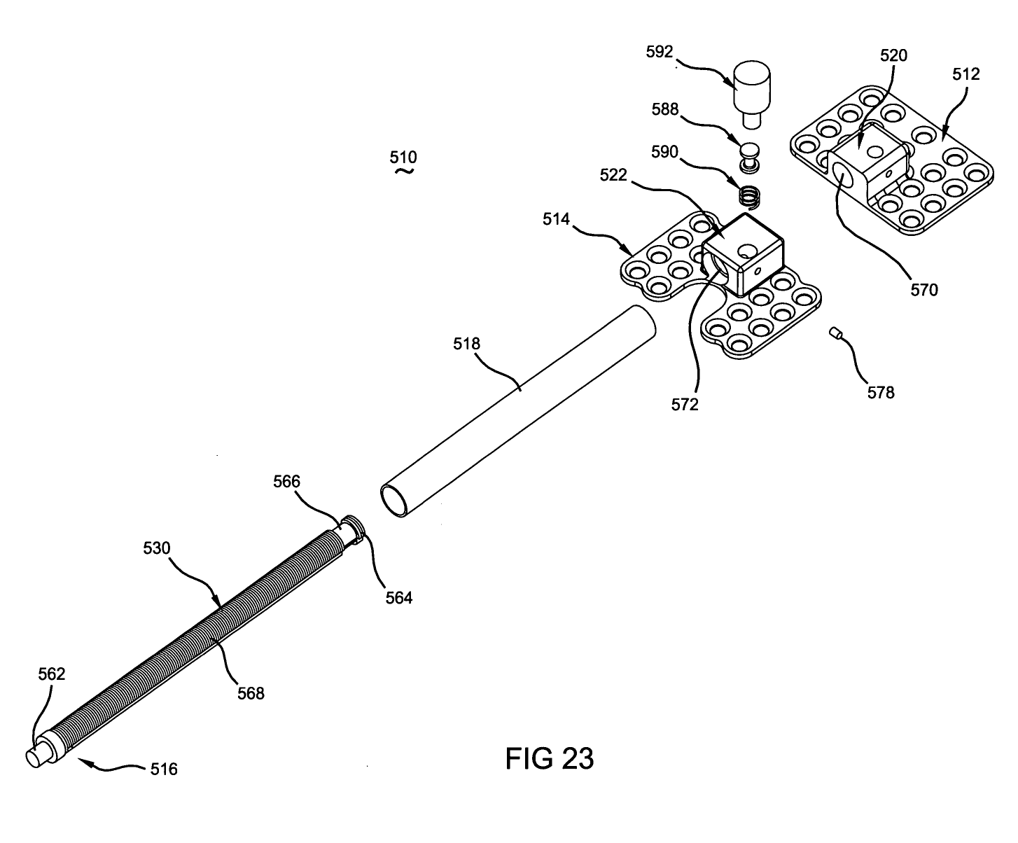

A flex tube 600 may also be provided in place of flexible sleeve 518 (FIG. 23) to preclude soft tissue from growing onto the drive screw 530. The flex tube 600 may be easily manipulated to an arcuate shape as shown in FIG. 26. The flex tube 600 does not have rebound or memory characteristics. As a result, the flex tube 600 does not have a tendency to resist movement or return to a previous orientation. The flex tube 600 may be used in combination with any bone distractor including those disclosed herein.

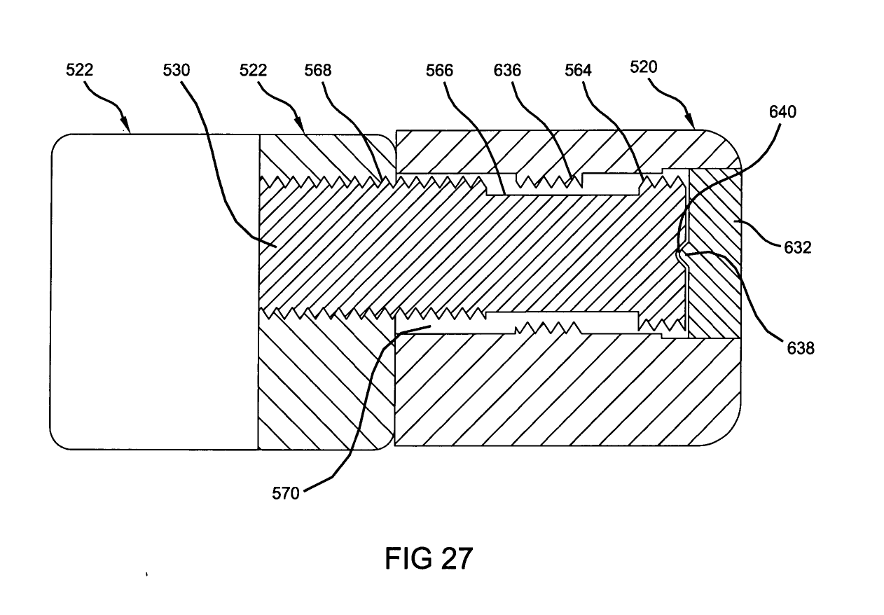

Turning now to FIG. 27, the tip 564 of the drive screw 530 is shown adjacent a stop 632. The stop 632 defines a nub 638 for nesting in a cavity 640 formed in the tip 564 of the drive screw 530. Threads 636 are defined on an inner bore of the central bridge portion 520. When the drive screw is turned clockwise, the threads 636 of the bridge portion 520 align with the non-threaded portion 566 and therefore, do not engage the drive screw 530. As illustrated, the tip 564 defines threads thereon. If the drive screw 530 is pulled leftward as illustrated in FIG. 27, the tip 564 engages the threads 636 precluding retraction of the drive screw 530 from the aperture 570. However, the drive screw 530 may be removed by initially pulling the drive screw 530 leftward until the threads on the tip 564 engage the threads 636 and then rotating the drive screw 530 in a counter-clockwise direction allowing the threads on the tip 564 to ride along the threads 636 of the bridge portion 520.

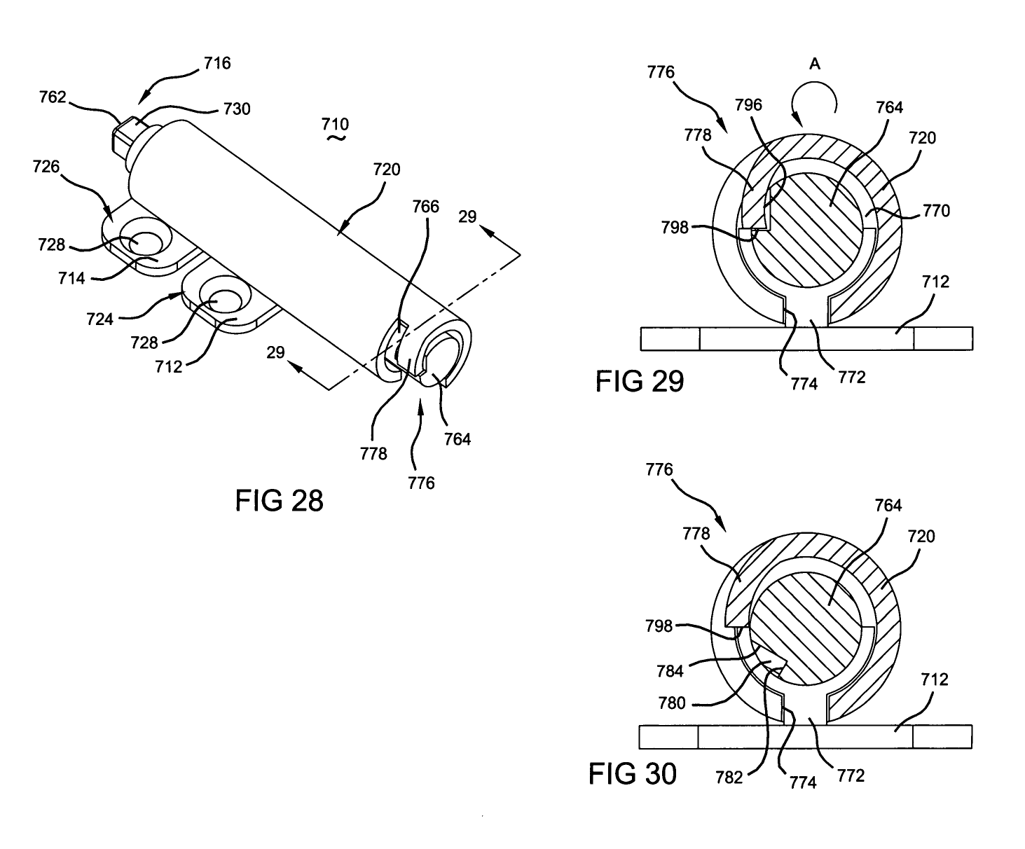

As illustrated in FIGS. 28-30, a bone distractor 710 may include a first affixation member 712, a second affixation member 714, and an adjustment assembly 716. The first affixation member 712 and the second affixation member 714 are moveable relative one another through the adjustment assembly 716.

The first affixation member 712 and the second affixation member 714 may include a cylindrical portion 720, and wing elements 724, 726, respectively. Wing elements 724, 726 may be generally planar and include multiple screw holes 728 operable to receive bone screws to secure wing elements 724, 726 to bone, wherein wing elements 724, 726 may lie flat adjacent to the applied bone surface.

The adjustment assembly 716 may include a drive screw 730 and a ratchet mechanism 576. The drive screw 730 may include a drive head 762 at one end and a tip 764 at an opposite end. Between the drive head 762 and the tip 764 is a non-threaded portion 766 and a threaded portion (not specifically shown). The drive head 762 may be driven by a tool. The tip 764 and non-threaded portion 766 may be received within an aperture 770 in the bridge portion 720.

The cylindrical portion 720 includes a ratchet mechanism 776. An end portion of the cylindrical portion 720 defines an integrally formed tang 778. The tip 764 and non-threaded portion 766 may be rotatably mounted in cylinder 720. A support frame 772 extends through a passage 774 formed in the cylindrical portion 720. The first affixation member 712 and may be selectively restricted from rotating in one direction by the ratchet mechanism 776. A groove 780 may include a first wall 782 and a second wall 784. The first wall 782 may be shorter than the second wall 784.

When the drive screw 730 is rotated in a first direction illustrated by Arrow A in FIG. 29, the tang engages second wall 784 of groove 780. The second wall 784 urges the tang 778 outboard (FIG. 30), thereby permitting rotation of the drive screw 730. When rotation in an opposite direction is attempted, the first wall 782 engages blocking surface 798 of the tang 778, thereby preventing rotation. Thus, rotation in the direction opposite Arrow A is not permitted due to blocking engagement of the first wall 782 and the blocking surface 798 of the tang 778.

The distractor 10, 110, 210, 310, 410, 510, 710 may be used in craniomaxillofacial applications as well as other orthopedic procedures. In use, the distractor may be temporarily affixed to the bone with first and second affixation members on opposite sides of the osteotomy, such as by bone screws. By rotating the drive screw incrementally and periodically, the two affixation members are driven apart, and hence the bones on either side of the osteotomy are distracted relative to the location of the osteotomy. A suitable tool for driving the drive screw, for example, is QC Torque-Limiting Patient Driver, available from Walter Lorenz Surgical, Inc., of Jacksonville, Fla. Rotation of the drive screw in the wrong direction, however, may be prevented by the ratchet mechanism, which limits the ability for a patient to manipulate the distractor improperly or for micromovements and force from surrounding soft tissue to move the two affixation members together along the drive screw.

The description is merely exemplary in nature and, thus, variations that do not depart from the teachings are intended to be within the scope of the teachings. Such variations are not to be regarded as a departure from the spirit and scope of the teachings.

What is claimed is:

1. A bone distractor for distracting bone on opposite sides of an osteotomy of said bone, comprising:
a first affixation member for affixation to said bone on one side of said osteotomy;
a second affixation member for affixation to said bone on another side of said osteotomy;
a rotatable member engaging said first affixation member and said second affixation member for distracting said first and second affixation members relative to each other in response to rotation of said rotatable member; and
a ratchet mechanism selectively engaging said rotatable member, said ratchet mechanism limiting rotation of said rotatable member in a first direction in one position and permitting rotation in said first direction in another position.

2. The bone distractor of claim 1, further comprising a release pin operably engaging said ratchet mechanism to move said ratchet mechanism between said one position and said another position.

3. The bone distractor of claim 2 wherein said ratchet mechanism includes a ratchet pin selectively engaged to said rotatable member in said one position.

4. The bone distractor of claim 3, further comprising a biasing member, said biasing member biasing said ratchet pin into said rotatable member in said one position.

5. The bone distractor of claim 4 wherein said rotatable member defines at least one groove longitudinally formed thereon, said groove defining a first wall and a second wall.

6. The bone distractor of claim 5 wherein said ratchet pin defines a cam surface, wherein said first wall engages said cam surface thereby forcing said ratchet pin against said biasing member and permitting rotation of said rotatable member in a second direction.

7. The bone distractor of claim 6 wherein said ratchet pin defines a blocking surface, wherein said second wall engages said blocking surface thereby precluding rotation of said rotatable member in said first direction.

8. The bone distractor of claim 1 wherein said rotatable member defines a threaded portion threadably communicating with one of said first and second affixation members and a non-threaded portion received in the other of said first and second affixation members, wherein said ratchet mechanism selectively engages said non-threaded portion of said rotatable member.

9. A bone distractor for distracting bone on opposite sides on an osteotomy of said bone, comprising:
a first affixation member for affixation to said bone on one side of said osteotomy;
a second affixation member for affixation to said bone on another side of said osteotomy;
a rotatable member engaging said first affixation member and said second affixation member for distracting said first and second affixation members relative to each other in response to rotation of said rotatable member; and
a ratchet mechanism selectively engaging said rotatable member, said ratchet mechanism limiting rotation of said rotatable member in a first direction and permitting rotation in a second direction.

10. The bone distractor of claim 9 wherein said rotatable member includes a groove and wherein said ratchet mechanism includes an engaging member selectively engaged with said groove.

11. The bone distractor of claim 10 wherein said engaging member rides on a first wall of said groove thereby deflecting said engaging member and permitting rotation in said second direction.

12. The bone distractor of claim 11 wherein said engaging member engages a second wall of said groove thereby precluding rotation of said engaging member in said first direction.

13. The bone distractor of claim 12 wherein said engaging member includes a spring having a free end adapted to deflect upon engaging said groove and rotation in said second direction.

14. The bone distractor of claim 12 wherein said engaging member includes a ratchet pin defining a cam surface, wherein said groove engages said cam surface thereby forcing said ratchet pin against a biasing member and permitting rotation of said rotatable member in said second direction.

15. The bone distractor of claim 9 wherein said rotatable member defines a threaded portion threadably communicating with one of said first and second affixation members and a non-threaded portion received in the other of said first and second affixation members, wherein said ratchet mechanism selectively engages said non-threaded portion of said rotatable member.

16. The bone distractor of claim 15 wherein said rotatable member defines a second threaded portion and wherein said other of said first and second affixation members defines a third threaded portion.

17. The bone distractor of claim 16 wherein said rotatable member is selectively removable from said other of said first and second affixation members upon rotation of said rotatable member in a first direction and engagement of said second and third threaded portions.

18. The bone distractor of claim 9, further comprising a flexible member having a first end extending from a distal end of said rotatable member and a second end defining an engagement surface, wherein said flexible member transmits rotational force applied at said engagement surface to said rotatable member.

19. The bone distractor of claim 18 wherein said flexible member defines a series of discontinuities and a corresponding series of links between adjacent discontinuities wherein adjacent links of said series of links are adapted to provide lateral displacement at said series of discontinuities in response to force applied to said flexible member.

20. The bone distractor of claim 19 wherein said flexible member is comprised of biocompatible metal.

21. A bone distractor for distracting bone on opposite sides of an osteotomy of said bone, comprising:
a first affixation member for affixation to said bone on one side of said osteotomy;
a second affixation member for affixation to said bone on another side of said osteotomy;
a rotatable member engaging said first affixation member and said second affixation member for distracting said first and second affixation members relative to each other in response to rotation of said rotatable member; and
a longitudinal extension coupled to said rotatable member and adapted to transmit torque to said rotatable member, said longitudinal extension having a series of interlocking links.

22. The bone distractor of claim 21 wherein said interlocking links define interlocking dovetail portions.

23. The bone distractor of claim 22 wherein said interlocking dovetail portions are adapted to provide lateral displacement between adjacent links.

24. The bone distractor of claim 23 wherein said interlocking links are adapted to permit said longitudinal extension to be placed in an arcuate orientation.

25. The bone distractor of claim 21 wherein a proximal end of said longitudinal extension defines an interface head adapted to communicate with a drive tool.

26. The bone distractor of claim 21 wherein said longitudinal extension is cannulated.

27. The bone distractor of claim 21 wherein said longitudinal extension is comprised of biocompatible metal.

28. The bone distractor of claim 27 wherein said biocompatible metal is selected from the group consisting of titanium, stainless steel and cobalt chrome alloy.

29. The bone distractor of claim 21 wherein said longitudinal extension is comprised of biocompatible plastic.

30. The bone distractor of claim 29 wherein said biocompatible plastic is resorbable.
